(19) United States
(12) Patent Application Publication
(10) Pub. No.: US 2006/0140868 A1
(43) Pub. Date: Jun. 29, 2006

(54) Method of preparation of biomagnetic nanoparticles coated with a noble metal layer

(76) Inventors: Stephanie Grancharov, New York, NY (US); Stephen O'Brien, New York, NY (US); Glenn Held, Pelham, NY (US); Christopher Bruce Murray, Ossining, NY (US)

Correspondence Address: BAKER & BOTTS, 30 ROCKEFELLER PLAZA, 44TH FLOOR, NEW YORK, NY, 10112, US

(21) Appl. No.: 11/156,740

(22) Filed: Jun. 20, 2005

Related U.S. Application Data
(63) Continuation of application No. PCT/US02/40550, filed on Dec. 19, 2002.

(51) Int. Cl. A61K 49/06; A61K 9/28

(52) U.S. Cl. 424/9.32; 427/2.14; 977/930

(57) ABSTRACT

A method for the preparation of magnetic nanoparticles coated with a noble metal is described. The method includes providing a first mixture containing a first non-polar organic solvent and uncoated magnetic nanoparticles. The first mixture is mixed with a second mixture containing a second non-polar organic solvent and ions comprising the noble metal to form a third mixture. The third mixture is mixed with an organic ligand or a fourth mixture containing an organic ligand to form a fifth mixture. The fifth mixture is reacted with a sixth mixture containing a reducing agent to form a seventh mixture containing the monodispersed magnetic nanoparticles coated with the noble metal. The monodispersed magnetic coated nanoparticles may be separated from the seventh mixture by adding a polar organic solvent or a mixture of polar organic solvents in which the nanoparticles are insoluble.

### BACKGROUND OF THE INVENTION

1. Field of the Invention

This invention relates to a method for the preparation of magnetic nanoparticles. More particularly, this invention relates to a method for the preparation of monodispersed magnetic nanoparticles coated with a noble metal.

2. Background Information

The synthesis of magnetic nanoparticles is of great interest because magnetic nanoparticles may be introduced into a wide range of cellular or cell-free biological systems. Application of an external magnetic field can then be used to guide the placement of magnetic nanoparticles inside a biological system to a specific region in the system. This is a particularly useful technique for guiding and assembling nanoparticles having biologically active molecules bound to their surface prior to introduction of the nanoparticles into the biological system. In addition, the magnetic nanoparticles modify the local magnetic field at nuclei of atoms of the biological system in the vicinity of the nanoparticles. The change in the local magnetic field may then be detected by NMR spectroscopy of the nuclei of the biological systems in which the magnetic nanoparticles have been introduced to obtain information on the development of these biological systems. Accordingly, several methods have been developed for the preparation of magnetic nanoparticles within a narrow size range. For example, U.S. Pat. No. 6,262,129 describes methods for preparing size selected nanoparticles of transition metals in organic solvents. Related synthetic approaches to the synthesis of iron nanoparticles have been described by Suslick, K. S., Fang, M. and Hyeon, T., Journal of the American Chemical Society, Vol. 118, No. 47(1996), p. 11960, and by Park, S.-J., et al., Journal of the American Chemical Society, Vol. 122, No. 35 (2000), p. 8581. All these methods involve the formation of metal nanoparticles by reduction of ions of the metal through thermal decomposition, sonofication, or chemical reducing agents. The final size of the nanoparticles is governed by the presence of organic surfactants, such as oleic acid, in the reaction medium. Typically, the nanoparticles obtained are composed of a magnetic core surrounded by organic ligands.

The preparation of a magnetic nanoparticle coated with a noble metal overlayer is of particular interest since the overlayer is likely to reduce the degradation of the magnetic core, thereby significantly enhancing the durability of such nanoparticles and their usefulness as a means to monitor the development of biological systems as discussed above. The synthesis of gold nanoparticles in toluene has been achieved by Brust, M., et al., Journal of the Chemical Society, Chemical Communications Vol. 7 (1994), p. 801, by a method involving the use of a phase transfer agent to transfer gold ions from an aqueous phase to the toluene phase, which is immiscible with the aqueous phase. Other organic solvents may also be used, as demonstrated by Korgel, B. A and Fitzmaurice, D., Physics Review Letters, Vol. 80, No. 16 (1998), p. 3531, and the final particle size and the nature of the stabilizing organic ligand may also be varied, as shown by Leff, D. V., Brandt, L. and Heath, J. R., Langmuir, Vol. 12, No. 20 (1996), p. 4723. A synthesis of gold coated particles using the reverse micelle technique, which is well known to produce particles in the micrometer regime, has been published by Lin, J., et al., Journal of Solid State Chemistry, Vol. 159, No. 1 (2001), p. 26. However, this approach provides nanoparticles that do not have a uniform size distribution. In particular, as shown by Carpenter, E., Journal of Magnetism and Magnetic Materials, Vol. 159, No. 1 (2001), pp. 26-31, the approach of Lin et al. gives nanoparticles in which the diameter standard deviation is up to twice the average value of the diameter. Therefore, the nanoparticles of Lin et al. are not monodispersed, where the term “monodispersed” is defined to mean that the average diameter of the nanoparticles has a standard deviation of 15% or less.

There is therefore a need in the art for a simple and effective method to produce noble metal coated magnetic nanoparticles having a narrow size distribution.

### SUMMARY OF THE INVENTION

The aforementioned need is substantially met by the present invention, which in one aspect is a method for producing monodispersed magnetic nanoparticles coated with a noble metal. The method in one exemplary embodiment comprises providing a first mixture containing a first non-polar organic solvent and uncoated magnetic nanoparticles. The first mixture is mixed with a second mixture containing a second non-polar organic solvent and ions containing a noble metal to form a third mixture. The third mixture is mixed with an organic ligand or a fourth mixture containing an organic ligand to form a fifth mixture. The fifth mixture is mixed with a sixth mixture containing a reducing agent to form a seventh mixture in which monodispersed magnetic nanoparticles coated with the noble metal are formed.

The method in one exemplary embodiment comprises providing a first mixture containing a first non-polar organic solvent and uncoated magnetic nanoparticles. The first mixture is mixed with a second mixture containing a second non-polar organic solvent, an organic ligand and ions containing the noble metal to form a third mixture. The third mixture is mixed with a fourth mixture containing a reducing agent to form a fifth mixture in which monodispersed magnetic nanoparticles coated with the noble metal are formed.

The advantage of the method of the invention is that it produces monodispersed magnetic nanoparticles coated with a noble metal that have superior stability to degradation of the magnetic core in comparison to magnetic nanoparticles that are not coated. Therefore, the magnetic nanoparticles coated with a noble metal prepared by the method of the invention can be used advantageously as magnetic markers in biological systems.

### BRIEF DESCRIPTION OF THE DRAWINGS

FIG. 1 shows schematically a prior art method for the preparation of uncoated iron nanoparticles.

FIG. 2A depicts a TEM image of 5 nm uncoated iron nanoparticles.

FIG. 2B depicts a TEM image of 7 nm nanoparticles of iron coated with gold.

FIG. 3A depicts a plot of the magnetic moment of iron nanoparticles coated with gold versus temperature for zero-field cooled (▪) and field cooled (●) particles.

FIG. 3B depicts a magnetic moment hysteresis curve of iron nanoparticles coated with gold at a temperature of 5° K.

### DETAILED DESCRIPTION OF THE PREFERRED EMBODIMENTS

As used herein, the term “monodispersed magnetic nanoparticles coated with a noble metal” means magnetic nanoparticles coated with a noble metal in which the standard deviation in the diameter of the nanoparticles is 15%.

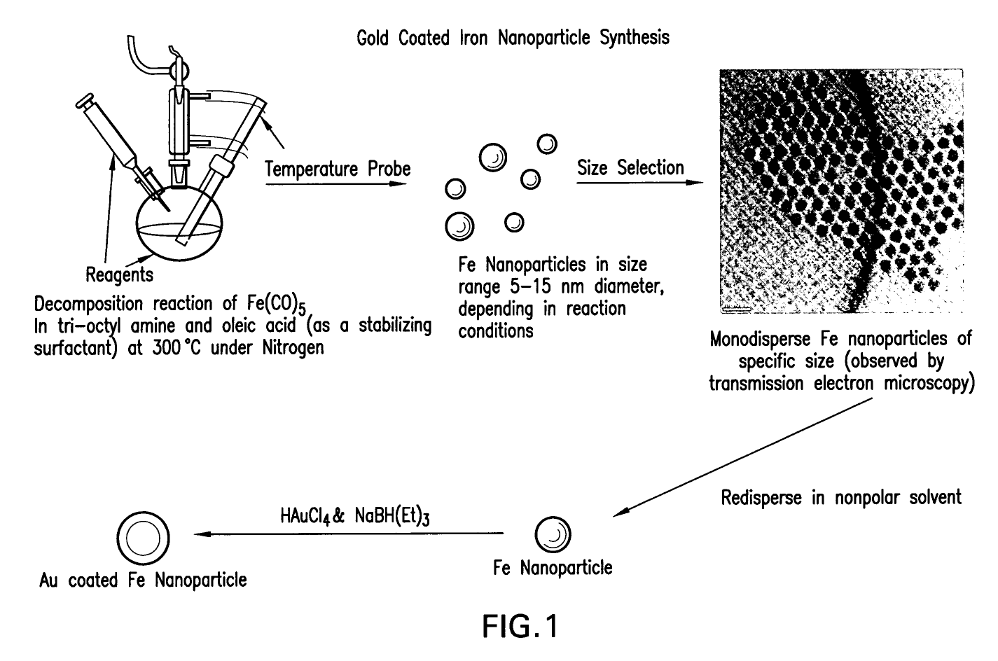

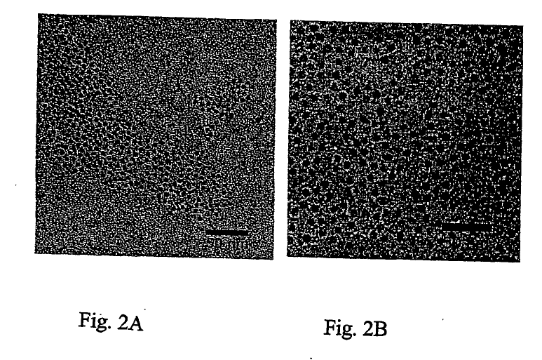

Uncoated magnetic nanoparticles are advantageously formed by thermal decomposition of a compound of a magnetic transition metal, sonochemical decomposition of a compound of a magnetic transition metal, or reduction of a compound of a magnetic transition metal with a reducing agent. Pentacarbonyl compounds, such as iron pentacarbonyl and cobalt pentacarbonyl, are especially advantageous magnetic transition metal compounds for this purpose. Each of these reactions is advantageously carried out in a mixture containing the compound of the magnetic transition metal and an organic solvent. Thermal decomposition of a compound of the magnetic transition metal in solution is an especially suitable method of forming the uncoated nanoparticles. For example, iron pentacarbonyl (Fe(CO)₅) may be decomposed to form iron nanoparticles according to the procedure described below and shown schematically in FIG. 1. This procedure is related to the procedures of Suslick et al., above, Park et al., above, and Hyeon, T., Lee, S. S., Park, J., Chung, Y and Na, H. B., “Synthesis of Highly Crystalline and Monodisperse Magehemite Nanocrystallites Without a Size Selection Process,” Journal of the American Chemical Society, Vol. 123, No. 51 (2001), pp. 12798-12801. However, the procedure shown in FIG. 1 differs from Suslick et al., Park et al., and Hyeon et al. in the solvent used and in the molar ratio of surfactant to iron pentacarbonyl, as discussed further below. According to the procedure shown in FIG. 1, a solution 101 containing an organic solvent having a boiling point of at least about 300° C., iron pentacarbonyl, and a surfactant such as oleic acid is heated in a nitrogen atmosphere from about 25° C. to about 300° C. and maintained at about 300° C. for about one hour in the nitrogen atmosphere to form iron nanoparticles. Advantageously, the organic solvent having a boiling point of at least about 300° C. is trioctyl amine or dioctyl ether and the molar ratio of surfactant to iron pentacarbonyl ranges from about 0.3:1 to about 40:1. Addition to the resulting mixture of a polar organic solvent, such as anhydrous ethanol, causes precipitation of the iron nanoparticles. The nanoparticles are separated from the mixture by centrifugation. The nanoparticles may be further purified by dispersing the nanoparticles in a first non-polar organic solvent such as, for example, toluene, hexane, or octane, to form a dispersion, and filtering the dispersion through a 0.05 μm filter. This procedure yields a mixture containing the first non-polar organic solvent and uncoated magnetic iron nanoparticles. The average diameter of the nanoparticles 103 varies from about 5 nm to about 10 nm and to about 15 nm, respectively, when the surfactant and iron pentacarbonyl are in a molar ratio of (0.3-0.6):1, (1.5-2.0): 1, and (3-4):1. In each case, the average diameter has a standard deviation of between 5% and 10% before the precipitation step and of less than 5% after the precipitation step. FIG. 2A shows a transmission electron microscope (TEM) image of iron nanoparticles obtained by depositing the nanoparticles from a dispersion of the nanoparticles in toluene onto a TEM grid. The nanoparticles are deposited dropwise from a syringe or suitable transfer vessel that allows microliter sized deposition. The toluene is then allowed to evaporate from the grid. The grid is preferably placed in a vacuum chamber for complete removal of the toluene. Additional control over the size distribution of the nanoparticles may be achieved though a plurality of cycles of precipitation and redissolution of the nanoparticles, as described in U.S. Pat. No. 6,262,129, herein incorporated by reference in its entirety; in Sun, S. and Murray, C. B., Journal of Applied Physics, Vol. 85, No. 8 (1999), p. 4325; and in Murray, C. B., et al., IBM Journal of Research and Development, Vol. 45 (2001), p. 47. A similar procedure may also be used to prepare magnetic nanoparticles containing pure iron oxide in the gamma phase (maghemite), as described by Hyeon, T. Lee, S. S., Park, J., Chung, Y., and Na, H. B., “Synthesis of Highly Crystalline and Monodisperse Maghemite Nanocrystallites Without a Size Selection Process,” above.

A similar procedure may be used to prepare magnetic nanoparticles containing iron and iron (III) oxide. This is achieved by heating and maintaining the solution containing an organic solvent, iron pentacarbonyl, and a surfactant in an atmosphere of air. The nanoparticles formed by this procedure contain both iron and iron (III) oxide, where iron (III) oxide is formed primarily on the surface of the nanoparticles. The nanoparticles containing iron and iron (III) oxide are then separated from the reaction mixture according to the procedure discussed above for the iron nanoparticles.

In one embodiment of the invention, a mixture containing a first and second non-polar organic solvents, uncoated magnetic nanoparticles, an organic ligand and a compound that dissociates to form ions containing the noble metal is prepared as follows. A mixture containing a second non-polar organic solvent and ions containing the noble metal is first prepared. This mixture is advantageously prepared by mixing an aqueous solution containing ions containing the noble metal with a solution containing the second non-polar organic solvent and a phase transfer agent. The second non-polar organic solvent may be selected from the group consisting of benzene, toluene, xylene, mesitylene, hexane, pentane, heptane, octane, dodecane, dioctyl ether, dibutyl ether and diethyl ether. Suitable phase transfer agents include cationic phase transfer agents such as alkylammonium halides and anionic phase transfer agents such as alkali metal carboxylates. The aqueous solution, which is initially colored due to the presence of the ions containing the noble metal, and the solution containing the second non-polar organic solvent and the phase transfer agent are shaken together for a time sufficient for the aqueous phase to become clear and colorless, after which the aqueous phase and the organic phase are allowed to separate. The aqueous phase typically becomes clear and colorless after about one hour, indicating that the ions containing the noble metal have been transferred into the second non-polar organic solvent to form the mixture containing the second non-polar organic solvent and the ions containing the noble metal. The mixture containing the second non-polar organic solvent and the ions containing the noble metal is separated from the aqueous phase by decantation and is then mixed with the mixture containing the uncoated magnetic nanoparticles and the first non-polar organic solvent to form a mixture containing the first and second non-polar organic solvents, the uncoated magnetic nanoparticles, and the ions containing the noble metal. Advantageously, the ions containing the noble metal are ions containing gold. In one particularly advantageous embodiment of the invention, the phase transfer catalyst is tetraoctylammonium bromide (TOAB, (C₈H₁₇)₄NBr), the first and second non-polar organic solvents are both toluene, and the ions containing the noble metal are tetrachloroaurate ions, which are provided by a tetrachloroaurate compound such as, for example, hydrogen tetrachloroaurate trihydrate (HAuCl₄.3H₂O). The mixture containing the first and second non-polar organic solvents, the uncoated magnetic nanoparticles, the organic ligand and the ions containing the noble metal is then prepared by mixing the mixture containing the first and second non-polar organic solvents, the uncoated magnetic nanoparticles, and the ions containing the noble metal with an organic ligand or a mixture containing an organic ligand. Suitable ligands include alkylthiols and alkylamines. Dodecanethiol is an especially advantageous ligand. The molar ratio of the organic ligand to the compound that dissociates to form ions containing the noble metal ranges from about 1:15 to about 1:5.

In another embodiment of the invention, the mixture containing the first and second non-polar organic solvents, the uncoated magnetic nanoparticles, the organic ligand and the ions containing the noble metal is prepared as follows. A mixture containing the second non-polar organic solvent, the organic ligand and a compound that dissociates to form ions containing the noble metal is first prepared. This mixture is advantageously prepared by mixing an aqueous solution containing ions containing the noble metal with a solution containing the second non-polar organic solvent, the organic ligand and a phase transfer agent. The aqueous solution and the solution containing the second non-polar organic solvent, the organic ligand and the phase transfer agent are shaken together for a time sufficient for the aqueous phase to become clear and colorless, indicating a formation of a mixture in the organic phase containing the second non-polar organic solvent, the organic ligand and the ions containing the noble metal. This mixture in the organic phase is separated from the aqueous phase by decantation and is then mixed with the mixture containing the uncoated magnetic nanoparticles and the first non-polar organic solvent to form the mixture containing the first and second non-polar organic solvents, the uncoated magnetic nanoparticles, the organic ligand and the ions containing the noble metal.

Without wishing to be bound by any mechanism or theory, it is believed that the magnetic nanoparticles in the presence of a ligand are surrounded by a “shell” containing molecules of the organic ligand bound non-covalently to the nanoparticle. Formation of the ligand shell acts to control the nanoparticle size by stabilizing the nanoparticle and preventing flocculation. The size of the nanoparticles also depends on the nature and concentration of the ligands.

The mixture containing the first and second non-polar organic solvents, the uncoated magnetic nanoparticles, the ions containing the noble metal and the organic ligand is then subjected to vigorous stirring after mixing with a mixture containing a reducing agent to form a mixture in which monodispersed magnetic nanoparticles coated with the noble metal are formed. The mixture containing a reducing agent is advantageously selected from a solution of sodium borohydride (NaBH₄) in water, a solution of SUPERHYDRIDE® (lithium triethylborohydride LiBH(C₂H₅)₃) in tetrahydroflran, and a solution of sodium triethylborohydride (NaBH(C₂H₅)₃) in tetrahydrofuran. A preferred mixture containing a reducing agent is a solution of sodium borohydride in water. This solution is added to the mixture containing the first and second non-polar organic solvents, the uncoated magnetic nanoparticles, the ions containing the noble metal and the organic ligand, and the resulting mixture is stirred for a period sufficient for the reduction of the ions containing the noble metal to occur. Without wishing to be bound by any mechanism or theory, it is believed that in the reduction of tetraaurochlorate ions with borohydride, the formation of gold takes place according to Equation (1) below:
6BH₄⁻+2AuCl₄⁻→6BH₃+2Au+3H₂+8Cl⁻ (1)

The ions containing the noble metal are reduced to the noble metal, which coats the magnetic nanoparticles, displacing the ligands during formation of the coating. If the mixture containing the reducing agent is a mixture containing water, the water defines an aqueous phase and the first and second organic solvents define an organic phase which is immiscible with the aqueous phase and separates from the aqueous phase once stirring is stopped. The monodispersed coated nanoparticles remain in the organic phase.

The organic phase may contain solid impurities which are removed by filtration or centrifugation. To separate the monodispersed coated nanoparticles from the organic phase, a polar organic solvent in which the nanoparticles are insoluble, such as, for example, an alcohol, may then be added to the organic phase to cause the nanoparticles to precipitate. The precipitation step may take place in the presence of a magnetic field created by a pole of a magnet placed at the based of the reaction flask. The magnetic field induces alignment of the nanoparticles during precipitation and applies an attractive force on the nanoparticles towards the bottom of the reaction flask to enhance the precipitation of the nanoparticles from the organic phase. The strength of the field is preferably about 0.2 Tesla. The monodispersed coated nanoparticles are then separated from the organic phase. Advantageously, the separation step is a centrifugation step. The nanoparticles may be further purified by dispersing the nanoparticles in a third non-polar organic solvent, such as toluene or hexane, to form a dispersion. The nanoparticles may then be separated from the third non-polar organic solvent through centrifugation.

By repeating the process of addition of the polar organic solvent to cause precipitation of the monodispersed magnetic coated nanoparticles, separating the coated nanoparticles from the organic phase, and redispersing the precipitated coated nanoparticles in the third non-polar organic solvent, the monodispersity of the monodispersed magnetic coated nanoparticles may be further improved, as described in U.S. Pat. No. 6,262,129 for uncoated magnetic nanoparticles. FIG. 2B shows a TEM image of 7 nm gold-coated iron nanoparticles obtained from the above procedure.

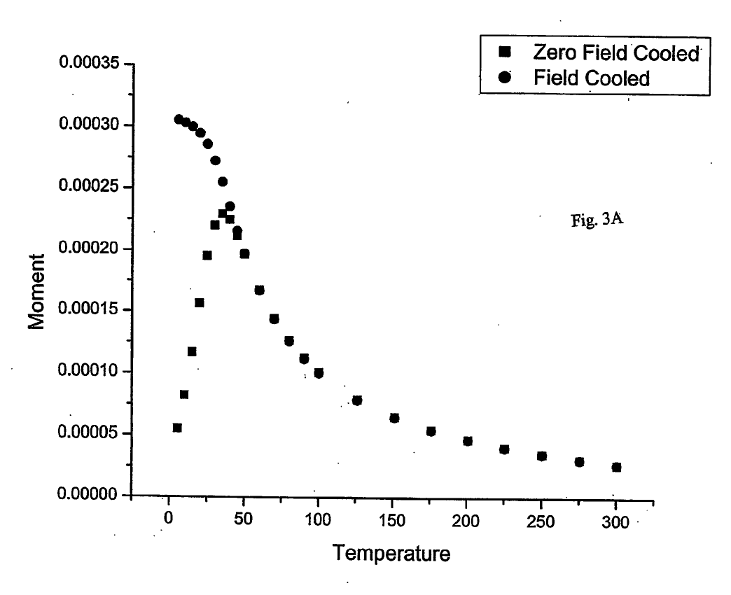

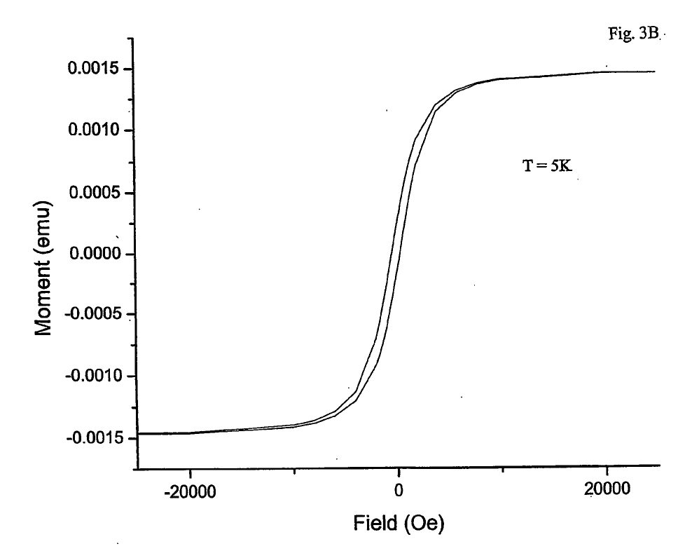

The iron nanoparticles coated with gold were characterized magnetically by measuring the magnetic moment of the nanoparticles as a function of temperature for zero-field cooled and field cooled particles, as shown in FIG. 3A, and by a magnetic moment hysteresis curve of the nanoparticles at a temperature of 5° K, shown in FIG. 3B. The magnetic moment of the nanoparticles was measured using a superconducting quantum interference device (SQUID). The zero-field cooled magnetic moment measurements were taken by first cooling a sample of iron nanoparticles coated with gold in the absence of a magnetic field from 300° K to 5° K A field of 200 Oesterd (Oe) was then applied to the sample and the magnetic moment was measured at temperatures between 5° K and 300° K. The field cooled magnetic moment measurements were taken by cooling the sample from 300° K to 5° K with the field of 200 Oe field applied to the sample. The magnetic moment was then measured at temperatures between 5° K and 300° K. The magnetic behavior of the nanoparticles illustrated in FIGS. 3A and 3B shows that the nanoparticles are nanoparticles having a magnetic iron core and a coating of gold.

The present invention is described in more detail with reference to the examples below, which, however, should be understood not to limit the invention in any way.

### EXAMPLE 1

### Preparation of Uncoated Iron Nanoparticles

A mixture of 28 mL oleic acid and 3 mL octyl ether was heated to 100° C. in a flask while the flask was being purged with nitrogen gas. Thereafter, 0.8 mL of Fe(CO)₅ were added to the mixture and the resulting mixture was heated to 300° C. The mixture was refluxed for 1 hour and allowed to cool to room temperature. The mixture was then centrifuged. A solid residue was formed which was separated from the liquid phase and discarded. The liquid phase containing ethanol and octyl ether was treated with five 10 mL aliquots of ethanol. After treatment with each aliquot of ethanol the resulting mixture was centrifuged to give a solid residue of uncoated iron nanoparticles which was separated from the liquid phase containing ethanol and octyl ether. Each of the five solid residues obtained from the five treatments with an aliquot of alcohol was dispersed in 10 mL of hexane to obtain a dispersion of uncoated iron nanoparticles in hexane. The resulting five dispersions of uncoated iron nanoparticles in hexane were combined to form one dispersion of uncoated iron nanoparticles in hexane.

### EXAMPLE 2

### Preparation of Iron Nanoparticles Coated with Gold

Four milliliters of the dispersion of uncoated iron nanoparticles in hexane obtained from Example 1 were exposed to a stream of nitrogen gas to cause evaporation of the solvent and give dry uncoated iron nanoparticles. The nanoparticles were suspended in 4 mL of toluene in a flask. A mixture of 0.023 g of HAuCl₄ and 15 mL of water was mixed with a mixture of 0.12 g TOAB and 5 mL of toluene and the resulting mixture was shaken until the aqueous phase was clear and colorless. The toluene and aqueous phases were allowed to separate. The resulting toluene phase containing gold ions was added to the flask containing the uncoated iron nanoparticles suspended in toluene. One tenth of a milliliter of a mixture of 0.15 mL of dodecanethiol and 16 mL toluene was added to the flask containing the uncoated iron nanoparticles suspended in toluene and the gold ions. 2 mL of a mixture of 0.75 mL of a 1.0 M solution of sodium triethylborohydride in tetrahydrofuran or toluene and 29.5 mL of toluene was added to the flask containing the uncoated iron nanoparticles suspended in toluene, the gold ions, and the dodecanethiol, and the resulting mixture was stirred for about 1 hour. The mixture was allowed to settle, and the liquid phase separated from unreacted solid material. The mixture was then centrifuged, and the resulting precipitate was discarded. The liquid phase was treated with five 10 mL aliquots of ethanol. After treatment with each aliquot of ethanol the resulting mixture was centrifuged to give a solid residue of iron nanoparticles coated with gold which was separated from the liquid phase. Each of the five solid residues obtained from the five treatments with an aliquot of alcohol was treated with 10 mL of hexane to obtain a dispersion of iron nanoparticles coated with gold in hexane. The resulting five dispersions of iron nanoparticles coated with gold in hexane were combined to form one dispersion of iron nanoparticles coated with gold in hexane.

### EXAMPLE 3 (PREDICTIVE)

### Preparation of Iron Nanoparticles Coated with Gold

Four milliliters of the dispersion of uncoated iron nanoparticles in hexane obtained from Example 1 are exposed to a stream of nitrogen gas to cause evaporation of the solvent and give dry uncoated iron nanoparticles. The nanoparticles are then suspended in 4 mL of toluene in a flask. A mixture of 0.023 g of HAuCl₄ and 15 mL of water is mixed in a flask with a mixture of 0.12 g TOAB, 0.01 mL of dodecanethiol and 5 mL of toluene and shaken until the aqueous phase is clear and colorless. The toluene and aqueous phases are then allowed to separate. The resulting toluene phase containing gold ions and dodecanethiol is added to the flask containing the uncoated iron nanoparticles suspended in toluene. Two milliliters of a mixture of 0.75 mL of sodium triethylborohydride and 29.5 mL of toluene are added to the flask containing the uncoated iron nanoparticles suspended in toluene, the gold ions, and the dodecanethiol, and the resulting mixture is stirred for about 1 hour. The mixture is allowed to settle and the liquid phase separated from unreacted solid material. The mixture is then centrifuged and the resulting precipitate is discarded. The liquid phase is treated with five 10 mL aliquots of ethanol. After treatment with each aliquot of ethanol the resulting mixture is centrifuged to give a solid residue containing iron nanoparticles coated with gold which is separated from the liquid phase. Each of the five solid residues resulting from the five treatments with an aliquot of alcohol is treated with 10 mL of hexane to obtain a dispersion of iron nanoparticles coated with gold in hexane. The resulting five dispersions of iron nanoparticles coated with gold in hexane are combined to form one dispersion of iron nanoparticles coated with gold in hexane.

It should be understood that various changes and modifications to the examplary embodiments described herein will be apparent to those skilled in the art. Such changes and modifications can be made without departing from the spirit and scope of this invention, the scope being defined by the appended claims.

We claim:

1. A method for making monodispersed magnetic nanoparticles coated with a noble metal, the method comprising:
(a) providing a first mixture containing a first non-polar organic solvent and uncoated magnetic nanoparticles;
(b) mixing the first mixture with a second mixture containing a second non-polar organic solvent and ions comprising the noble metal to form a third mixture;
(c) mixing the third mixture with one of an organic ligand and a fourth mixture containing an organic ligand to form a fifth mixture; and
(d) mixing the fifth mixture with a sixth mixture containing a reducing agent to form a seventh mixture in which the monodispersed magnetic nanoparticles coated with the noble metal are formed.

2. The method of claim 1, wherein the uncoated magnetic nanoparticles are formed by a reaction selected from the group consisting of:
(i) thermally decomposing a compound of a magnetic transition metal;
(ii) sonochemically decomposing a compound of a magnetic transition metal; and
(iii) chemically reducing a compound of a magnetic transition metal.

3. The method of claim 2, wherein the uncoated magnetic nanoparticles are formed by thermally decomposing a compound of the magnetic transition metal.

4. The method of claim 3, wherein thermally decomposing the compound of the magnetic transition metal comprises:
(A) forming a mixture comprising the compound of the magnetic transition metal and a solvent having a boiling point of at least about 300° C.; and
(B) maintaining the mixture at about 300° C. for about one hour.

5. The method of claim 4, wherein the solvent having a boiling point of at least about 300° C. is dioctyl ether and the mixture formed in step (A) further comprises a surfactant.

6. The method of claim 5, wherein the surfactant and the compound of the magnetic transition metal are in a molar ratio ranging from about 0.3:1 to about 40:1.

7. The method of claim 3, wherein the compound of the magnetic transition metal is selected from the group consisting of compounds of iron and compounds of cobalt.

8. The method of claim 7, wherein the compound of the magnetic transition metal is selected from the group consisting of iron pentacarbonyl and cobalt pentacarbonyl.

9. The method of claim 7, wherein the compound of the magnetic transition metal is a compound of iron and the uncoated magnetic nanoparticles are iron nanoparticles.

10. The method of claim 7, wherein the compound of the magnetic transition metal is a compound of iron and the uncoated magnetic nanoparticles are nanoparticles comprising iron and iron (III) oxide.

11. The method of claim 1, wherein the second mixture is formed by mixing an aqueous solution containing the ions comprising the noble metal with a solution comprising the second non-polar organic solvent and a phase transfer agent.

12. The method of claim 1, wherein the noble metal is gold and the ions comprising the noble metal are ions comprising gold.

13. The method of claim 12, wherein the ions comprising gold are tetrachloroaurate ions.

14. The method of claim 1, wherein the organic ligand is selected from the group consisting of alkylthiols and alkylamines.

15. The method of claim 1, wherein the sixth mixture containing the reducing agent is selected from the group consisting of a mixture containing sodium borohydride and water, a mixture containing lithium triethylborohydride and tetrahydrofuran, and a mixture containing sodium triethylborohydride and tetrahydrofuran.

16. The method of claim 1, wherein the sixth mixture containing the reducing agent also contains water, whereby the seventh mixture contains water defining an aqueous phase and contains the first and second organic solvents defining an organic phase which is immiscible with the aqueous phase, the method further comprising:
(e) separating the organic phase from the aqueous phase in the seventh mixture, the monodispersed magnetic nanoparticles coated with the noble metal being formed in the organic phase of the seventh mixture;
(f) adding a polar organic solvent to the organic phase of the seventh mixture to cause the monodispersed magnetic nanoparticles coated with the noble metal formed therein to precipitate; and
(g) separating the precipitated monodispersed magnetic nanoparticles coated with the noble metal from the organic phase of the seventh mixture.

17. The method of claim 16, wherein step (f) further comprises applying a magnetic field to the organic phase of the seventh mixture so as to enhance precipitation of the magnetic nanoparticles coated with the noble metal therein.

18. A method for making monodispersed magnetic nanoparticles coated with a noble metal, the method comprising:
(a) providing a first mixture comprising a first non-polar organic solvent and uncoated magnetic nanoparticles;
(b) mixing the first mixture with a second mixture containing a second non-polar organic solvent, an organic ligand and ions comprising the noble metal to form a third mixture; and
(c) mixing the third mixture with a fourth mixture containing a reducing agent to form a fifth mixture in which the monodispersed magnetic nanoparticles coated with the noble metal are formed.

19. The method of claim 18, wherein the uncoated magnetic nanoparticles are formed by a reaction selected from the group consisting of
(i) thermally decomposing a compound of a magnetic transition metal;
(ii) sonochemically decomposing a compound of a magnetic transition metal; and
(iii) chemically reducing a compound of a magnetic transition metal.

20. The method of claim 19, wherein the uncoated magnetic nanoparticles are formed by thermally decomposing a compound of the magnetic transition metal.

21. The method of claim 20, wherein thermally decomposing the compound of the magnetic transition metal comprises:
(A) forming a mixture comprising the compound of the magnetic transition metal and a solvent having a boiling point of at least about 300° C.; and
(B) maintaining the mixture at about 300° C. for about one hour.

22. The method of claim 21, wherein the solvent having a boiling point of at least about 300° C. is dioctyl ether and the mixture formed in step (A) further comprises a surfactant.

23. The method of claim 22, wherein the surfactant is oleic acid.

24. The method of claim 20, wherein the compound of the magnetic transition metal is selected from the group consisting of compounds of iron and compounds of cobalt.

25. The method of claim 24, wherein the compound of the magnetic transition metal is selected from the group consisting of iron pentacarbonyl and cobalt pentacarbonyl.

26. The method of claim 24, wherein the compound of the magnetic transition metal is a compound of iron and the uncoated magnetic nanoparticles are iron nanoparticles.

27. The method of claim 24, wherein the compound of the magnetic transition metal is a compound of iron and the uncoated magnetic nanoparticles are nanoparticles comprising iron and iron (III) oxide.

28. The method of claim 18, wherein the second mixture is formed by mixing an aqueous solution containing the ions comprising the noble metal with a solution comprising the second non-polar organic solvent, the organic ligand and a phase transfer agent.

29. The method of claim 18, wherein the noble metal is gold and the ions comprising the noble metal are ions comprising gold.

30. The method of claim 29, wherein the ions comprising gold are tetrachloroaurate ions.

31. The method of claim 18, wherein the organic ligand is selected from the group consisting of alkylthiols and alkylamines.

32. The method of claim 18, wherein the mixture comprising the reducing agent is selected from a mixture comprising sodium borohydride and water, a mixture comprising lithium triethylborohydride and tetrahydrofuran, and a mixture comprising sodium triethylborohydride and tetrahydrofuran.
